(19) United States
(12) Patent Application Publication
(10) Pub. No.: US 2016/0183828 A1
(43) Pub. Date: Jun. 30, 2016

(54) ELECTRONIC APPARATUS FOR ESTABLISHING PREDICTION MODEL BASED ON ELECTROENCEPHALOGRAM

(71) Applicant: I-SHOU UNIVERSITY, KAOHSIUNG CITY (TW)

(72) Inventors: Chen-Sen Ouyang, KAOHSIUNG CITY (TW); Lung-Chang Lin, KAOHSIUNG CITY (TW); Ching-Tai Chiang, KAOHSIUNG CITY (TW); Rei-Cheng Yang, KAOHSIUNG CITY (TW); Rong-Ching Wu, KAOHSIUNG CITY (TW); Hui-Chuan Wu, KAOHSIUNG CITY (TW)

(21) Appl. No.: 14/819,445

(22) Filed: Aug. 6, 2015

(30) Foreign Application Priority Data
Dec. 30, 2014 (TW) 103146255

(51) Int. Cl. A61B 5/04; A61B 5/00; A61B 5/0476

(52) U.S. Cl. CPC A61B 5/04012; A61B 5/0476; A61B 5/4094; A61B 5/4848

(57) ABSTRACT

An electronic apparatus for establishing prediction model based on electroencephalogram (EEG). The electronic apparatus is configured for: acquiring an EEG signal segment related to an epilepsy patient; dividing each EEG signal into EEG components according to a predetermined window size; retrieving datasets corresponding to EEG features from the EEG components of each EEG signal segment; acquiring statistical feature values of each dataset of each EEG signal segment; determining a gain ratio of each of the statistical feature values of each EEG signal segment based on the statistical feature values corresponding to each of the EEG features; selecting specific statistical feature values from the statistical feature values according to the gain ratio of each of the statistical feature values of each EEG signal segment; establishing a prediction model based on the specific statistical feature values of the epilepsy patient.

### CROSS-REFERENCE TO RELATED APPLICATION

This application claims the priority benefit of Taiwan application serial no. 103146255, filed on Dec. 30, 2014. The entirety of the above-mentioned patent application is hereby incorporated by reference herein and made a part of this specification.

### BACKGROUND OF THE INVENTION

1. Field of the Invention

The invention relates to an electronic apparatus, and more particularly, relates to an electronic apparatus for establishing prediction model based on electroencephalogram.

2. Description of Related Art

Epilepsy is the most common chronic disease in pediatric neurology. Among epileptic children, 60% to 70% of patients can be well-controlled by antiepileptic drug (AED), and this epilepsy type is known as a well-controlled epilepsy. On the other hand, an epilepsy type that is not controllable by AED is known as a refractory epilepsy. Because therapies for the well-controlled epilepsy and the refractory epilepsy are different, if the epilepsy type of the patients may be predicted accurately, the patients can receive more appropriate therapy earlier.

As one of common therapies for improving epilepsy conditions, a music therapy mainly focused on letting the patients listen to music, such as Mozart K.448. However, not every patient shows improvements on the conditions after listening to the music. Therefore, if it can be accurately predicted whether the music therapy can help to improve the epilepsy conditions a patient, that patient can still receive more appropriate therapy earlier.

In addition, one of influences brought to the patient by the epilepsy is that a time point at onset is random. Accordingly, if whether an epilepsy seizure state of the patient belongs to an inter-ictal state or a pre-ictal state (i.e., a state when the epilepsy is about to attack) can be accurately predicted, the patient and people nearby may be able to respond quickly, so as to reduce negative impacts caused by the onset.

### SUMMARY OF THE INVENTION

Accordingly, the invention is directed to an electronic apparatus for establishing prediction model based on electroencephalogram (EEG). The electronic apparatus is capable of locating appropriate statistical feature values from an EEG of an epilepsy patient based on specific mechanisms, and establishing a prediction model based on the statistical feature values. The prediction model is used for predicting an epilepsy type of an epilepsy patient, a therapeutic efficacy of a music therapy to the epilepsy patient and an epilepsy seizure state in response to different mechanisms.

The invention provides an electronic apparatus for establishing prediction model based on electroencephalogram, which includes a storage unit and a processing unit. The storage unit records a plurality of modules. The processing unit is coupled to the modules and configured to access and execute the modules. The modules include a first acquiring module, a dividing module, a retrieving module, a second acquiring module, a determining module, a selecting module and an establishing module. The first acquiring module acquires at least one EEG signal segment related to a first epilepsy patient via a plurality of detection electrodes. Each EEG signal segment includes a plurality of EEG signals corresponding to a plurality of channels, and each of the channels is corresponding to one of a plurality of bipolar montages. The dividing module divides each of the EEG signals into a plurality of EEG components according to a predetermined window size. The retrieving module retrieves a plurality of datasets corresponding to a plurality of EEG features from the EEG components of each EEG signal segment. The second acquiring module acquires a plurality of statistical feature values of each of the datasets of each EEG signal segment. The determining module determines a gain ratio of each of the statistical feature values of each EEG signal segment based on the statistical feature values corresponding to each of the EEG features. The selecting module selects specific statistical feature values from the statistical feature values according to the gain ratio of each of the statistical feature values of each EEG signal segment. The establishing module establishes a prediction model based on the specific statistical feature values of the first epilepsy patient.

Based on the above, the electronic apparatus proposed according to the embodiments of the invention is capable of locating the specific statistical feature values from the EEG of the first epilepsy patient, and accordingly establishing the predication model for predicting the epilepsy type of the epilepsy patient, the therapeutic efficacy of the music therapy to the epilepsy patient and the epilepsy seizure state.

To make the above features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram illustrating an electronic apparatus for establishing prediction model based on electroencephalogram according to an embodiment of the invention.

FIG. 2 is a flowchart illustrating a method for establishing prediction model based on electroencephalogram according to the first embodiment of the invention.

FIG. 3A is a schematic diagram illustrating an EEG signal segment according to an embodiment of the invention.

FIG. 3B illustrates a plurality of feature values corresponding to an EEG feature according to the embodiment of FIG. 3A.

FIG. 3C is a schematic diagram illustrating a calculation of the statistical feature values according to the embodiment of FIG. 3B.

FIG. 4 is a flowchart illustrating a method for predicting the epilepsy type based on the prediction model according to an embodiment of the invention.

FIG. 5 is a flowchart illustrating a method for predicting the therapeutic efficacy of the music therapy based on the prediction model according to an embodiment of the invention.

FIG. 6 is a flowchart illustrating a method for predicting the epilepsy seizure state based on the prediction model according to an embodiment of the invention.

### DETAILED DESCRIPTION

Reference will now be made in detail to the present preferred embodiments of the invention, examples of which are illustrated in the accompanying drawings. Wherever possible, the same reference numbers are used in the drawings and the description to refer to the same or like parts.

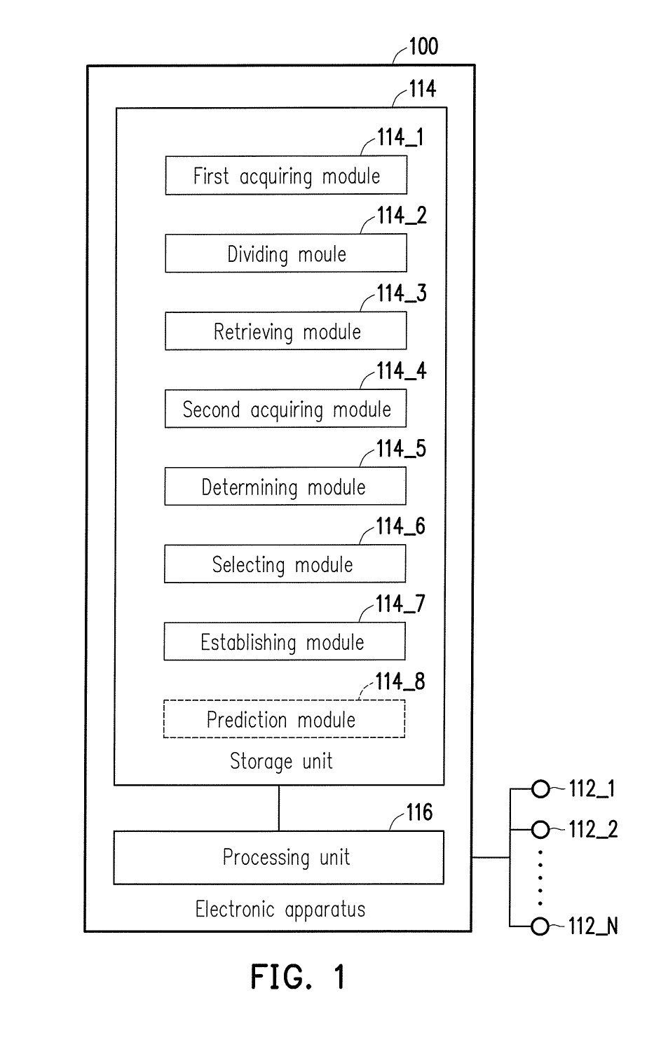

FIG. 1 is a schematic diagram illustrating an electronic apparatus for establishing prediction model based on electroencephalogram according to an embodiment of the invention. An electronic apparatus 100 is, for example, a medical apparatus capable of measuring an electroencephalogram (EEG) of an epilepsy patient and accordingly providing related physiological data as references for medical personnel. Such medical apparatus may be disposed with, for example, a display for displaying said physiological data and a user interface (e.g., push buttons or a touch screen) for the medical personnel to operate. Further, the electronic apparatus 100 may also be a device for processing the EEG, such as a personal computer, a work station, a server, a smart phone, a tablet computer, a notebook computer and so on.

In the present embodiment, the electronic apparatus 100 includes detection electrodes 112_1 to 112_N (where N is a positive integer), a storage unit 114 and a processing unit 116. The detection electrodes 112_1 to 112_N may be attached onto a scalp of the epilepsy patient in order to measure the EEG of the epilepsy patient. Specifically, the detection electrodes 112_1 to 112_N are corresponding to a plurality of bipolar montages, and the bipolar montages are corresponding to a plurality of channels. Aforesaid channels may be, for example, F3-C3, F4-C4, C3-T3, C4-T4, T3-O1, T4-O2, O1-C3 and O2-C4. Persons of ordinary skill in the art should be understood that English alphabets as included in each of the channels are corresponding to the placements of the detection electrodes 112_1 to 112_N on the scalp, but the implementation of the invention is not limited thereto.

The storing unit 114 may be, for example, a memory, a hard disk or other devices capable of storing data for recording a plurality of program codes or modules. The processing unit 116 can be a processor for general purposes, a processor for special purposes, a conventional processor, a data signal processor, a plurality of microprocessors, one or more microprocessors, controllers, microcontrollers and Application Specific Integrated Circuit (ASIC) which are combined to a core of the digital signal processor, a Field Programmable Gate Array (FPGA), any other integrated circuits, a state machine, a processor based on Advanced RISC Machine (ARM) and similar products.

In the present embodiment, the processing unit 116 may access and execute a first acquiring module 114_1, a dividing module 114_2, a retrieving module 114_3, a second acquiring module 114_4, a determining module 114_5, a selecting module 114_6 and an establishing module 114_7 in the storage unit 114 in order to execute a method for establishing prediction model based on electroencephalogram as proposed by the invention.

As mentioned above, the prediction model is used for predicting the epilepsy type, the therapeutic efficacy of the music therapy to the epilepsy patient and the epilepsy seizure state (hereinafter, collectively known as a condition feature of the patient) in response to different establishment mechanisms. Accordingly, in order to describe aforesaid mechanisms more clearly, the method for establishing various prediction models are described below by a first embodiment, a second embodiment and a third embodiment, respectively.

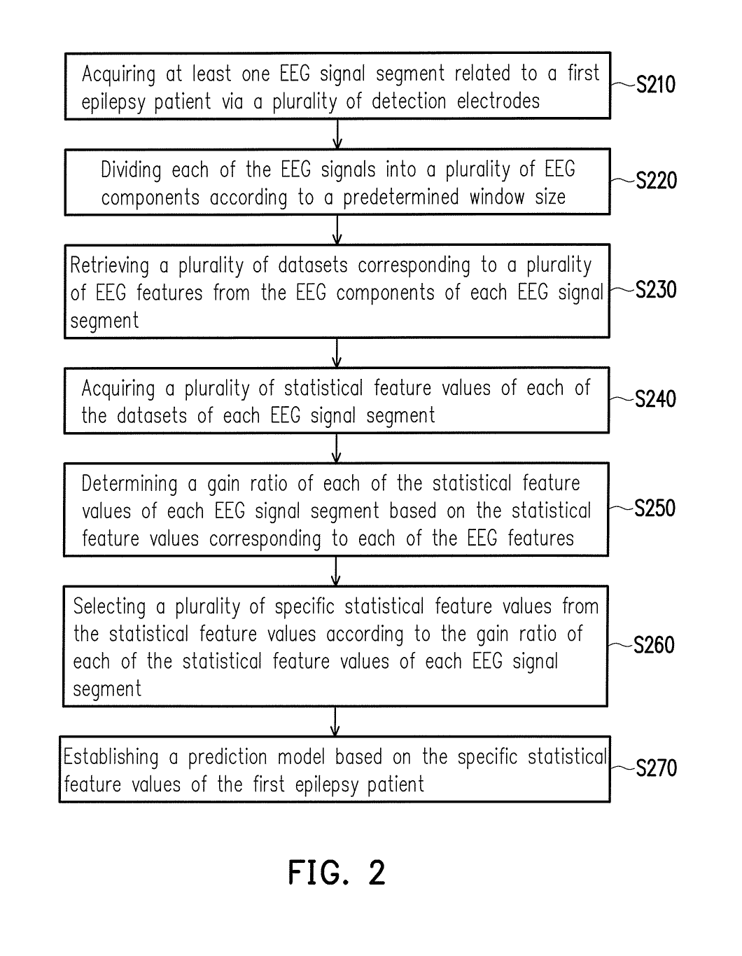

FIG. 2 is a flowchart illustrating a method for establishing prediction model based on electroencephalogram according to the first embodiment of the invention. The method proposed by the present embodiment can be executed by the electronic apparatus 100 depicted in FIG. 1, and each steps of the present embodiment is described in detail with reference to each element depicted in FIG. 1.

In step S210, the first acquiring module 114_1 may acquire at least one EEG signal segment related to a first epilepsy patient via a plurality of detection electrodes 112_1 to 112_N. The first epilepsy patient is, for example, an i^{th} (where i is a positive integer) epilepsy patient among a plurality of epilepsy patients with a known epilepsy type. Further, in the present embodiment, said first epilepsy patient is not yet received an antiepileptic drug treatment. Subsequently, in step S220, the dividing module 114_2 may divide each of the EEG signals into a plurality of EEG components according to a predetermined window size (e.g., 5 seconds).

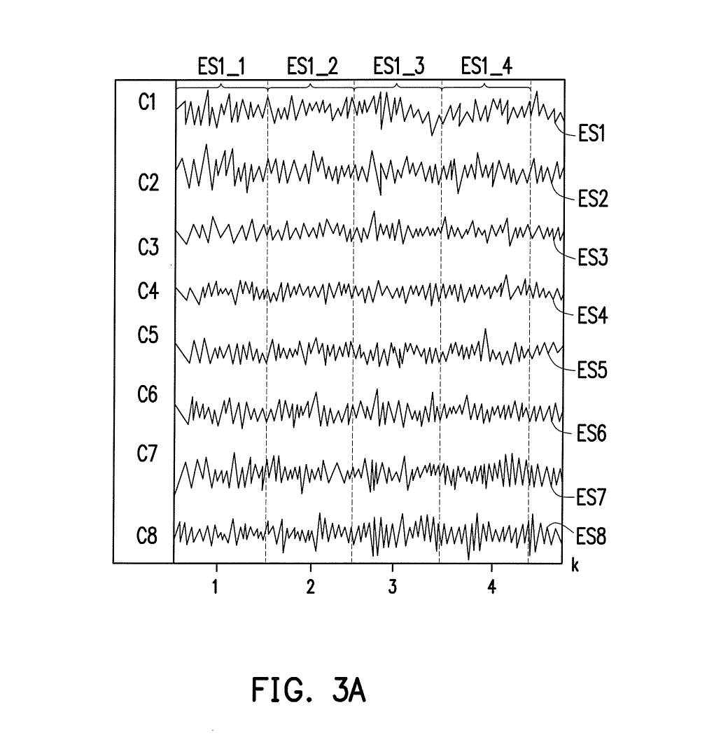

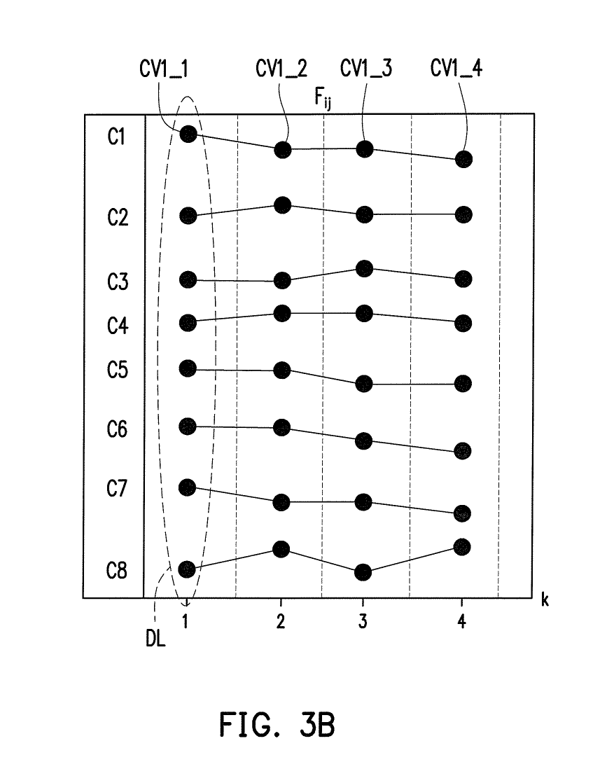

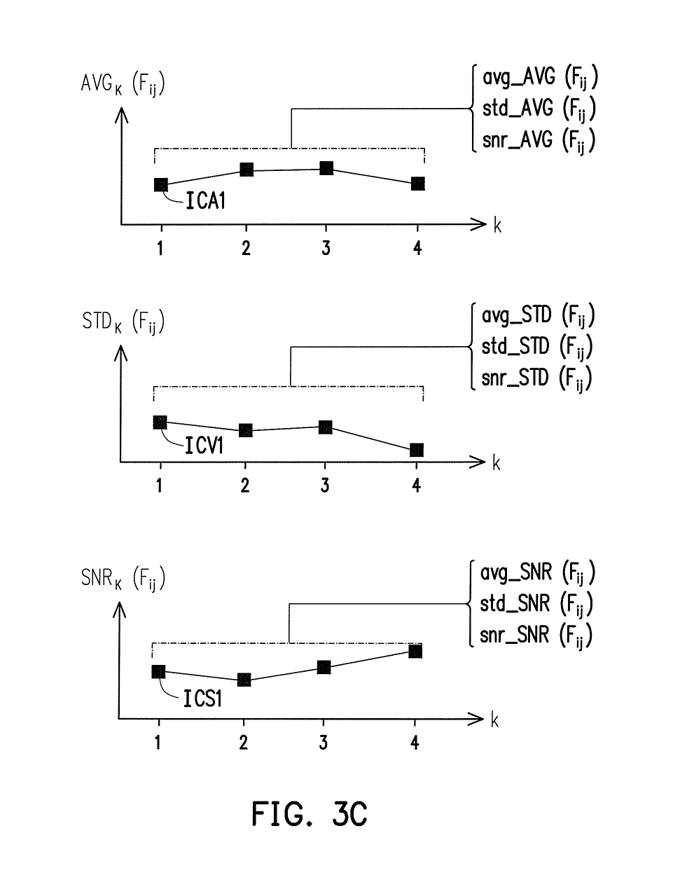

Referring to FIG. 3A, which is a schematic diagram illustrating an EEG signal segment according to an embodiment of the invention. In the present embodiment, an EEG signal segment Eᵢ is an EEG signal segment of the first epilepsy patient. It should be understood that, the EEG signal segment Eᵢ is, for example, an artifact-free signal acquired after performing an artifact eliminating mechanism on a raw EEG signal of the first epilepsy patient acquired by the first acquiring module 114_1.

As shown in FIG. 3A, the EEG signal segment Eᵢ includes EEG signals ES1 to ES8 corresponding to eight channels C1 to C8. Each of the EEG signals ES1 to ES8 includes a plurality of sampling values acquired by the first acquiring module 114_1 according to a sampling frequency (e.g., 200 Hz), and each of the EEG signals ES1 to ES8 includes a plurality of EEG components divided by the dividing module 114_2 according to the predetermined window size. Take the EEG signal ES1 as an example, the EEG signal ES1 is, for example, corresponding to the channel C1 and includes EEG components ES1_1 to ES1_4. In the present embodiment, the dividing module 1142 may divide each of the EEG signals ES1 to ES8 into four windows, but the implementation of the invention is not limited thereto.

Subsequently, in step S230, the retrieving module 114_3 may retrieve a plurality of datasets corresponding to a plurality of EEG features from the EEG components of each EEG signal segment.

In an embodiment, the EEG features include an auto regressive modeling error, a decorrelation time, an EEG energy, an approximate entropy, a sample entropy, a mobility, a relative power of a plurality of frequency bands, a spectral edge frequency, a spectral edge power, a plurality of moments and a plurality of energy of wavelet coefficients. The frequency bands are, for example, 0.1 to 4 Hz, 4 to 8 Hz, 8 to 15 Hz, 15 to 30 Hz, 30 to 2000 Hz, etc. The moments are, for example, mean, variance, skewness and kurtosis. The energy of wavelet coefficients are, for example, energy of Daubechies order 4 wavelet transform in decomposition levels 1 to 6, etc.

In an embodiment, the retrieving module 114_3 may retrieve feature values of each of the EEG features from each of the EEG components in FIG. 3A through a software package related to EEG analysis, such as EPILAB, and may represent the feature values as the corresponding dataset.

Referring to FIG. 3B, which illustrates a plurality of feature values corresponding to an EEG feature according to the embodiment of FIG. 3A. In the present embodiment, each of the feature values is corresponding to each of the EEG components shown in FIG. 3A in a one-to-one manner, and each of the feature values denotes a value of the EEG feature retrieved from the corresponding EEG component. For instance, feature values CV1_1 to CV1_4 are corresponding to the EEG components ES1_1 to ES1_4 in the one-to-one manner. In other words, assuming that the considered EEG features are the decorrelation times, the feature values CV1_1 to CV1_4 may denote values of the decorrelation times retrieved from the EEG components ES1_1 to ES1_4, respectively.

In an embodiment, an i^{th} dataset corresponding to a j^{th} (where j is a positive integer) EEG feature may be characterized by:

$${F}_{ij} = [ \begin{matrix}{f}_{ij} ( 1 , 1 ) & {f}_{ij} ( 1 , 2 ) & … & {f}_{ij} ( 1 , {n}_{i}^{′} ) \\ {f}_{ij} ( 2 , 1 ) & {f}_{ij} ( 2 , 2 ) & ⋯ & {f}_{ij} ( 2 , {n}_{i}^{′} ) \\ ⋮ & ⋮ & ⋱ & ⋮ \\ {f}_{ij} ( C , 1 ) & {f}_{ij} ( C , 2 ) & … & {f}_{ij} ( C , {n}_{i}^{′} )\end{matrix} ] , j = 1 , … , {E}_{f}$$

where C is an amount of the channels, E_{f} is an amount of the EEG features, fᵢⱼ(l,k) is a feature value of a k^{th} EEG component of a l^{th} (where l is a positive integer) channel (C, E_{f}, l and k are positive integers). In nᵢ′=└nᵢ/(fₛ·W)┘, nᵢ is an amount of the sampling values, fₛ is the sampling frequency (e.g., 200 Hz), W is the predetermined window size (e.g., 5 seconds), and └•┘ is a floor function.

In the case where FIG. 3B is corresponding to the j^{th} EEG feature, the dataset of FIG. 3B may be characterized by:

$${F}_{ij} = [ \begin{matrix}{f}_{ij} ( 1 , 1 ) & {f}_{ij} ( 1 , 2 ) & … & {f}_{ij} ( 1 , 4 ) \\ {f}_{ij} ( 2 , 1 ) & {f}_{ij} ( 2 , 2 ) & ⋯ & {f}_{ij} ( 2 , 4 ) \\ ⋮ & ⋮ & ⋱ & ⋮ \\ {f}_{ij} ( 8 , 1 ) & {f}_{ij} ( 8 , 2 ) & … & {f}_{ij} ( 8 , 4 )\end{matrix} ]$$

Among which, fᵢⱼ(1,1) to fᵢⱼ(1,4) are corresponding to the feature values CV1_1 to CV1_4, respectively, but the implementation of the invention is not limited thereto.

Referring back to FIG. 2, in step S240, the second acquiring module 114_4 may acquire a plurality of statistical feature values of each of the datasets of each EEG signal segment.

In an embodiment, the statistical feature values of the i^{th} dataset corresponding to the j^{th} EEG feature include a plurality of average values, a plurality of standard deviations and a plurality of signal-to-noise ratios. In this case, the second acquiring module 114_4 may calculate a plurality of inter-channel average values, a plurality of inter-channel standard deviations and a plurality of signal-to-noise ratios of the i^{th} dataset corresponding to the j^{th} EEG feature, and calculate a plurality of average values over time, a plurality of standard deviations over time and a plurality of signal-to-noise ratios over time according to the inter-channel average values, the inter-channel standard deviations and the inter-channel signal-to-noise ratios.

In an embodiment, a k^{th} inter-channel average value among the inter-channel average values may be characterized by:

$${AVG}_{k} ( {F}_{ij} ) = \frac{1}{C} {\sum }_{l = 1}^{C} {f}_{ij} ( l , k ) .$$

A k^{th} inter-channel standard deviation among the inter-channel standard deviations may be characterized by:

$${STD}_{k} ( {F}_{ij} ) = \sqrt{\frac{1}{C} {\sum }_{l = 1}^{C} {( {f}_{ij} ( l , k ) - {AVG}_{k} ( {F}_{ij} ) )}^{2}} .$$

A k^{th} inter-channel signal-to-noise ratio among the inter-channel signal-to-noise ratios may be characterized by:

$${SNR}_{k} ( {F}_{ij} ) = \frac{{AVG}_{k} ( {F}_{ij} )}{{STD}_{k} ( {F}_{ij} )} .$$

In this case, a first average value, a second average value and a third average value among the average values may be respectively characterized by:

$$avg\_AVG ( {F}_{ij} ) = \frac{1}{{n}_{i}^{′}} {\sum }_{k = 1}^{{n}_{i}^{′}} {AVG}_{k} ( {F}_{ij} ) , \\ avg\_STD ( {F}_{ij} ) = \frac{1}{{n}_{i}^{′}} {\sum }_{k = 1}^{{n}_{i}^{′}} {STD}_{k} ( {F}_{ij} ) and$$

$$avg\_SNR ( {F}_{ij} ) = \frac{1}{{n}_{i}^{′}} {\sum }_{k = 1}^{{n}_{i}^{′}} {SNR}_{k} ( {F}_{ij} ) .$$

A first standard deviation, a second standard deviation and a third standard deviation among the standard deviations may be respectively characterized by:

$${std\_AVG}_{k} ( {F}_{ij} ) = \sqrt{\frac{1}{{n}_{i}^{′}} {\sum }_{k = 1}^{{n}_{i}^{′}} {( {AVG}_{k} ( {F}_{ij} ) - avg\_AVG ( {F}_{ij} ) )}^{2}} , \\ {std\_STD}_{k} ( {F}_{ij} ) = \sqrt{\frac{1}{{n}_{i}^{′}} {\sum }_{k = 1}^{{n}_{i}^{′}} {( {STD}_{k} ( {F}_{ij} ) - avg\_STD ( {F}_{ij} ) )}^{2}} and$$

$${std\_SNR}_{k} ( {F}_{ij} ) = \sqrt{\frac{1}{{n}_{i}^{′}} {\sum }_{k = 1}^{{n}_{i}^{′}} {( {SNR}_{k} ( {F}_{ij} ) - avg\_SNR ( {F}_{ij} ) )}^{2}} .$$

A first signal-to-noise ratio, a second signal-to-noise ratio and a third signal-to-noise ratio among the signal-to-noise ratios may be respectively characterized by:

$$snr\_AVG ( {F}_{ij} ) = \frac{avg\_AVG ( {F}_{ij} )}{std\_AVG ( {F}_{ij} )} , \\ snr\_STD ( {F}_{ij} ) = \frac{avg\_STD ( {F}_{ij} )}{std\_STD ( {F}_{ij} )} and$$

$$snr\_SNR ( {F}_{ij} ) = \frac{avg\_SNR ( {F}_{ij} )}{std\_SNR ( {F}_{ij} )} .$$

Referring to FIG. 3C, which is a schematic diagram illustrating a calculation of the statistical feature values according to the embodiment of FIG. 3B. In the present embodiment, the second acquiring module 114_4 may, for example, calculate an inter-channel average value ICA1 (i.e., AVGₖ(Fᵢⱼ)|ₖ₌₁), an inter-channel standard deviation ICV1 (i.e., STDₖ(Fᵢⱼ)|ₖ₌₁) and an inter-channel signal-to-noise ratio ICS1 (i.e., SNRₖ(Fᵢⱼ)|ₖ₌₁) based on each of the feature values within a dash-line box DL (e.g., a first window). After all of AVGₖ(Fᵢⱼ), STDₖ(Fᵢⱼ) and SNRₖ(Fᵢⱼ) corresponding to the four windows are calculated, the second acquiring module 114_4 may correspondingly calculate nine statistical feature values including avg_AVG(Fᵢⱼ), avg_STD(Fᵢⱼ), avg_SNR(Fᵢⱼ), std_AVGₖ(Fᵢⱼ), std_STDₖ(Fᵢⱼ), std_SNRₖ(Fᵢⱼ), snr_AVG(Fᵢⱼ), snr_STD(Fᵢⱼ) and snr_SNR(Fᵢⱼ).

In an embodiment, the statistical feature values calculated based on Fᵢⱼ may further be characterized by a global feature descriptor matrix related to Fᵢⱼ, which is:

$${GF}_{ij} = [ \begin{matrix}avg\_AVG ( {F}_{ij} ) & std\_AVG ( {F}_{ij} ) & snr\_AVG ( {F}_{ij} ) \\ avg\_STD ( {F}_{ij} ) & std\_STD ( {F}_{ij} ) & snr\_STD ( {F}_{ij} ) \\ avg\_SNR ( {F}_{ij} ) & std\_SNR ( {F}_{ij} ) & snr\_SNR ( {F}_{ij} )\end{matrix} ] .$$

In step S250, the determining module 114_5 may determine a gain ratio of each of the statistical feature values of each EEG signal segment based on the statistical feature values corresponding to each of the EEG features. Specifically, assuming that the amount of the considered EEG features is Q (where Q is a positive integer), an amount of the statistical feature values that can be calculated from one EEG signal segment is, for example, 9×Q.

In an embodiment, the determining module 114_5 may calculate the gain ratio of each of the 9×Q number of statistical feature values through data mining software such as Weka. It should be understood that, the operation mechanism and the related principles of Weka may refer to those cited in related documents, thus details regarding how to calculate the gain ratio of each of said 9×Q number of statistical feature values by the determining module 114_5 are omitted herein. Schematically speaking, as the gain ratio of one specific statistical feature value being greater, it indicates that the specific statistical feature value may contribute more in determining the epilepsy type.

Therefore, in step S260, the selecting module 114_6 may select a plurality of specific statistical feature values from the statistical feature values according to the gain ratio of each of the statistical feature values of each EEG signal segment. Specifically, the selecting module 114_6 may rank the gain ratio of each of the 9×Q number of statistical feature values in a descending order according to the gain ratio of each of the statistical feature values. Subsequently, the selecting module 114_6 may select a predetermined number (e.g., 10) of the top-ranked statistical feature values from the 9×Q number of statistical feature values of each EEG signal segment to serve as the specific statistical feature values. In other words, the specific statistical feature values are statistical feature values which contribute the most in determining the epilepsy type in one EEG signal segment.

Thereafter, in step S270, the establishing module 114_7 may establish a prediction model based on the specific statistical feature values of the first epilepsy patient. In an embodiment, the establishing module 114_7 may use the specific statistical feature values to train a classifier (e.g., a support vector machine (SVM)), so as to establish the prediction model. As mentioned above, the epilepsy type (the refractory epilepsy/the well-controlled epilepsy) of the first epilepsy patient is known. Accordingly, the establishing module 114_7 may use the epilepsy type of the first epilepsy patient and said specific statistical feature values as training data for the SVM (e.g., a v-SVM). Subsequently, the establishing module 114_7 may locate a hyperplane for discriminating the refractory epilepsy and the well-controlled epilepsy based on the EEG signal segment of the first epilepsy patient (with the known epilepsy type).

Although the foregoing embodiments use one first epilepsy patient to describe the method according to the embodiments of the invention, persons of ordinary skill in the art should be able to understand that the method according to the embodiments of the invention may also be applied to a plurality of the first epilepsy patient. Further, as the number of the first epilepsy patients increases, the training data for training the prediction model are also increased to improve an accuracy of the prediction model for predicting the epilepsy type.

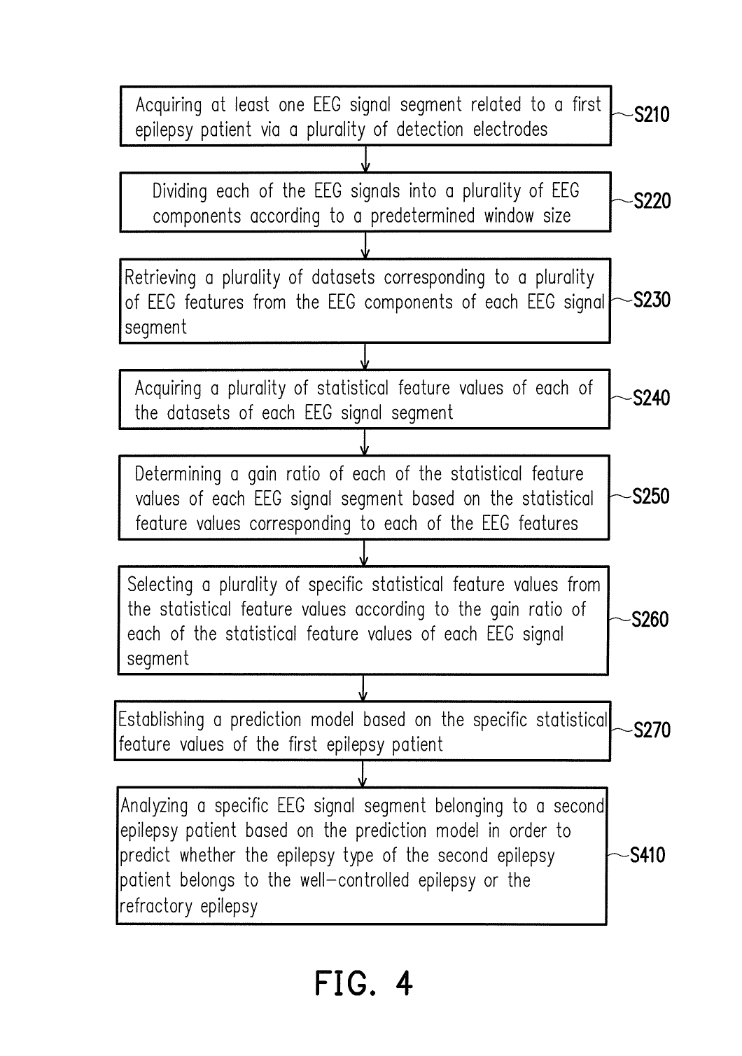

In other embodiments, the storage unit 114 may further include a prediction module 114_8. Referring to FIG. 4, which is a flowchart illustrating a method for predicting the epilepsy type based on the prediction model according to an embodiment of the invention. In the present embodiment, after steps S210 to S270 are executed, in step S410, the prediction module 114_8 may analyze a specific EEG signal segment belonging to a second epilepsy patient based on the prediction model in order to predict whether the epilepsy type of the second epilepsy patient belongs to the well-controlled epilepsy or the refractory epilepsy.

Specifically, the prediction module 114_8 may locate the specific statistical feature values from the specific EEG signal segment based on the teachings of FIG. 3A to FIG. 3C. Subsequently, the prediction module 114_8 may input the specific statistical feature values to the prediction model in order to classify specific statistical feature values through the hyperplane in the prediction model. Thereafter, the prediction module 114_8 may predict whether the second epilepsy patient belongs to the well-controlled epilepsy or the refractory epilepsy based on a classified result.

In brief, the electronic apparatus proposed in the embodiments of the invention is capable of locating the specific statistical feature values contributing in determining the epilepsy type from the EEG of the first epilepsy patient whose epilepsy type is known, and establishing the prediction model for predicting the epilepsy type based on the specific statistical feature values. In other words, the electronic apparatus proposed according to the embodiments of the invention provides an effective and quantized method for predicting the epilepsy type.

As mentioned above, in the embodiments of the invention, the prediction model for predicting the therapeutic efficacy of the music therapy to the epilepsy patient is further provided according to the second embodiment, which is described in detail as follows.

In the second embodiment, the electronic apparatus 100 may also execute steps S210 to S270 to establish the prediction model for predicting the therapeutic efficacy of the music therapy to the epilepsy patient.

However, one of differences between the second embodiment and the first embodiment is that the second embodiment considers whether the first epilepsy patient belongs to a first-type patient or a second-type patient. The first-type patient represents patients whose epilepsy condition is improvable by the music therapy, and the second-type patient represents patients whose epilepsy condition is not improvable by the music therapy.

Further, the at least one EEG signal segment of the first epilepsy patient (to whom whether the therapeutic efficacy of the music therapy is effective/ineffective is known) considered in the second embodiment includes two EEG signal segments. In the at least one EEG signal segment, a first EEG signal segment is corresponding to an EEG state of the first epilepsy patient before receiving the music therapy, and a second EEG signal segment is corresponding to the EEG state of the first epilepsy patient receiving the music therapy.

In such condition, an i^{th} dataset corresponding to a k^{th} EEG signal segment and a j^{th} EEG feature may be characterized by:

$${F}_{ij}^{( k )} = [ \begin{matrix}{f}_{ij}^{( k )} ( 1 , 1 ) & {f}_{ij}^{( k )} ( 1 , 2 ) & … & {f}_{ij}^{( k )} ( 1 , {n}_{i}^{( k )} ) \\ {f}_{ij}^{( k )} ( 2 , 1 ) & {f}_{ij}^{( k )} ( 2 , 2 ) & … & {f}_{ij} ( 2 , {n}_{i}^{( k )} ) \\ ⋮ & ⋮ & ⋱ & ⋮ \\ {f}_{ij}^{( k )} ( C , 1 ) & {f}_{ij}^{( k )} ( C , 2 ) & … & {f}_{ij}^{( k )} ( C , {n}_{i}^{( k )} )\end{matrix} ] , j = 1 , … , {E}_{f} , k = 1 , 2$$

where C is an amount of the channels, E_{f} is an amount of the EEG features, fᵢⱼ^{(k)}(l,m) is a m^{th} (m is a positive integer) EEG component of a l^{th} channel, nᵢ^{(k)}=└nᵢ/(fₛ·W)┘.

A second difference between the second embodiment and the first embodiment is that, the second acquiring module 114_4 must take in consideration of both Fᵢⱼ⁽¹⁾ and Fᵢⱼ⁽²⁾ when acquiring the statistical feature values in the second embodiment.

Specifically, for Fᵢⱼ⁽¹⁾, the second acquiring module 114_4 calculates a plurality of first inter-channel average values, a plurality of first inter-channel standard deviations and a plurality of first inter-channel signal-to-noise ratios of the i^{th} dataset corresponding to the first EEG signal segment and the j^{th} EEG feature according to the teachings of FIG. 3A to FIG. 3C. Subsequently, the second acquiring module 1144 calculates a plurality of first average values, a plurality of first standard deviations and a plurality of first signal-to-noise ratios according to the first inter-channel average values, the first inter-channel standard deviations and the first inter-channel signal-to-noise ratios.

Thereafter, the second acquiring module 114_4 may represent the first average values, the first standard deviations and the first signal-to-noise ratios by the corresponding global feature descriptor matrix (hereinafter, referred to as a first matrix).

On the other hand, for Fᵢⱼ⁽²⁾, the second acquiring module 114_4 calculates a plurality of second inter-channel average values, a plurality of second inter-channel standard deviations and a plurality of second inter-channel signal-to-noise ratios of the i^{th} dataset corresponding to the second EEG signal segment and the j^{th} EEG feature. Subsequently, the second acquiring module 114_4 calculates a plurality of second average values, a plurality of second standard deviations and a plurality of second signal-to-noise ratios according to the second inter-channel average values, the second inter-channel standard deviations and the second inter-channel signal-to-noise ratios.

Thereafter, the second acquiring module 114_4 may represent the second average values, the second standard deviations and the second signal-to-noise ratios by the corresponding global feature descriptor matrix (hereinafter, referred to as a second matrix).

In the present embodiment, a generalized correlation of the first matrix and the second matrix may be characterized by:

$${GF}_{ij}^{( k )} = [ \begin{matrix}avg\_AVG ( {F}_{ij}^{( k )} ) & std\_AVG ( {F}_{ij}^{( k )} ) & snr\_AVG ( {F}_{ij}^{( k )} ) \\ avg\_STD ( {F}_{ij}^{( k )} ) & std\_STD ( {F}_{ij}^{( k )} ) & snr\_STD ( {F}_{ij}^{( k )} ) \\ avg\_SNR ( {F}_{ij}^{( k )} ) & std\_SNR ( {F}_{ij}^{( k )} ) & snr\_SNR ( {F}_{ij}^{( k )} )\end{matrix} ] , k = 1 , 2.$$

In this case, the second acquiring module 114_4 may subtract the first matrix (i.e., GFᵢⱼ⁽¹⁾) from the second matrix (i.e., GFᵢⱼ⁽²⁾) in order to acquire a third matrix (i.e., GFᵢⱼ⁽²⁾-GFᵢⱼ⁽¹⁾) which includes the average values, the standard deviations and the signal-to-noise ratios.

Subsequently, the determining module 114_5 determines a gain ratio of each of the statistical feature values (i.e., each of elements in the third matrix) of each EEG signal segment based on the statistical feature values corresponding to each of the EEG features. Thereafter, the selecting module 114_6 may select a plurality of specific statistical feature values from the statistical feature values according to the gain ratio of each of the statistical feature values of each EEG signal segment. Then, the establishing module 114_7 may establish a prediction model based on the specific statistical feature values of the first epilepsy patient. Details regarding the determining module 114_5, the selecting module 114_6 and the establishing module 114_7 may refer to the descriptions in the first embodiment, which are not repeated hereinafter.

In brief, in the second embodiment, because the therapeutic efficacy (effective/ineffective) of the music therapy to the first epilepsy patient is known, the establishing module 144_7 may use the therapeutic efficacy of the music therapy to the first epilepsy patient and the specific statistical feature values corresponding to the first epilepsy patient as training data for the SVM (e.g., v-SVM). Subsequently, the establishing module 114_7 may locate a hyperplane for discriminating the therapeutic efficacy of the music therapy to the epilepsy patient based on the EEG signal segment of the first epilepsy patient.

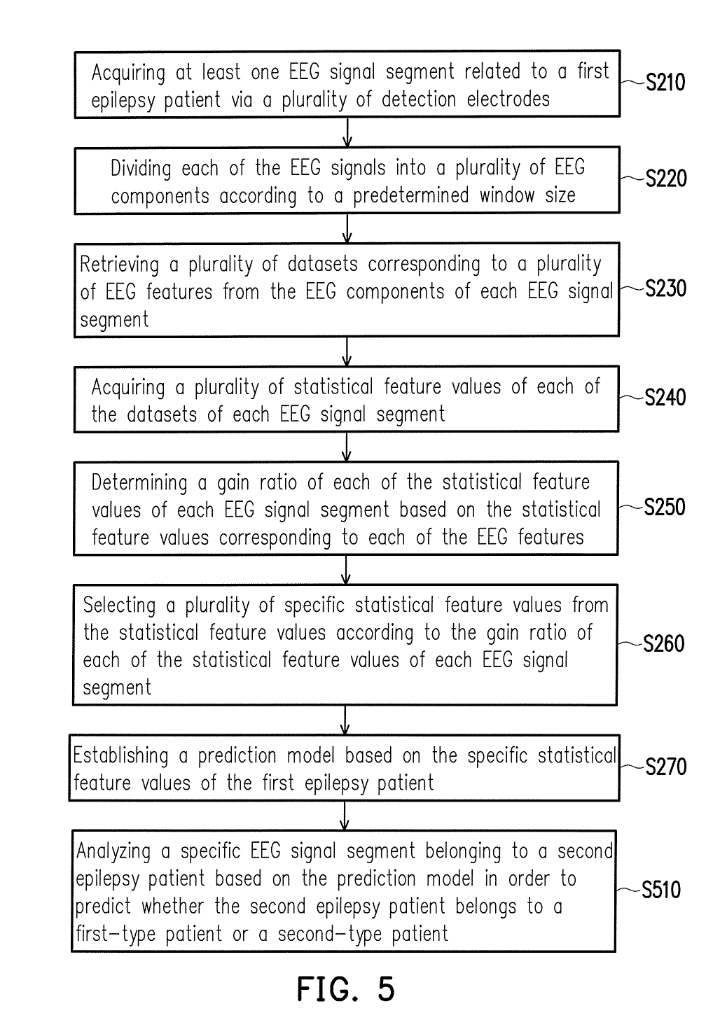

Referring to FIG. 5, which is a flowchart illustrating a method for predicting the therapeutic efficacy of the music therapy based on the prediction model according to an embodiment of the invention. In the present embodiment, after steps S210 to S270 are executed, in step S510, the prediction module 114_8 may analyze a specific EEG signal segment belonging to a second epilepsy patient based on the prediction model in order to predict whether the second epilepsy patient belongs to a first-type patient or a second-type patient.

Specifically, the prediction module 114_8 may locate the specific statistical feature values of the second epilepsy patient based on the above teachings. Subsequently, the prediction module 114_8 may input the specific statistical feature values to the prediction model in order to classify specific statistical feature values through the hyperplane in the prediction model. Thereafter, the prediction module 114_8 may predict whether the second epilepsy patient belongs to the first-type patient or the second-type patient based on a classified result.

In brief, the electronic apparatus proposed in the embodiments of the invention is capable of locating the specific statistical feature values contributing in determining whether the music therapy is effective from the EEG of the first epilepsy patient to whom the therapeutic efficacy of the music therapy is known, and establishing the prediction model for predicting the therapeutic efficacy of the music therapy based on the specific statistical feature values. In other words, the electronic apparatus proposed according to the embodiments of the invention provides an effective and quantized method for predicting the therapeutic efficacy of the music therapy.

As mentioned above, in the embodiments of the invention, the prediction model for predicting the epilepsy seizure state of the epilepsy patient is further provided according to the third embodiment, which is described in detail as follows.

In the third embodiment, the electronic apparatus 100 may also execute steps S210 to S270 to establish the prediction model for predicting the epilepsy seizure state of the epilepsy patient.

However, one of differences between the third embodiment and the first embodiment is that the first acquiring module 114_1 acquires the at least one EEG signal segment from the artifact-free signal based on a sliding window mechanism. Adjacent two EEG signal segments in the at least one EEG signal segment overlap with each other for a predetermined time interval (e.g., 20 seconds), and the sliding window mechanism is corresponding to a sliding window size (e.g., 30 seconds).

In the present embodiment, because the epilepsy seizure state reflected by each EEG signal segment on the first epilepsy patient is known, the electronic apparatus 100 is capable of establishing the corresponding prediction model based on each EEG signal segment.

In such condition, an i^{th} dataset corresponding to a j^{th} EEG signal segment and a k^{th} EEG feature may be characterized by:

$${F}_{ij}^{( k )} = [ \begin{matrix}{f}_{ij}^{( k )} ( 1 , 1 ) & {f}_{ij}^{( k )} ( 1 , 2 ) & … & {f}_{ij}^{( k )} ( 1 , \frac{SW}{W} ) \\ {f}_{ij}^{( k )} ( 2 , 1 ) & {f}_{ij}^{( k )} ( 2 , 2 ) & … & {f}_{ij} ( 2 , \frac{SW}{W} ) \\ ⋮ & ⋮ & ⋱ & ⋮ \\ {f}_{ij}^{( k )} ( C , 1 ) & {f}_{ij}^{( k )} ( C , 2 ) & … & {f}_{ij}^{( k )} ( C , \frac{SW}{W} )\end{matrix} ] , \\ k = 1 , … , {E}_{f} , j = 1 , … , {M}_{i}$$

wherein fᵢⱼ^{(k)}(l,m) is a feature value of a m^{th} EEG component of a l^{th} channel, Mᵢ is an amount of the at least one EEG signal segment, and SW is the sliding window size.

For each of Fᵢⱼ^{(k)}, the second acquiring module 114_4 is capable of calculating the corresponding statistical feature values according to the teachings of FIG. 3A to FIG. 3C. Subsequently, the determining module 114_5 determines a gain ratio of each of the statistical feature values (i.e., each of elements in the third matrix) of each EEG signal segment based on the statistical feature values corresponding to each of the EEG features. Thereafter, the selecting module 114_6 may select a plurality of specific statistical feature values from the statistical feature values according to the gain ratio of each of the statistical feature values of each EEG signal segment. Then, the establishing module 114_7 may establish a prediction model based on the specific statistical feature values of the first epilepsy patient. Details regarding the determining module 114_5, the selecting module 114_6 and the establishing module 114_7 may refer to the descriptions in the first embodiment, which are not repeated hereinafter.

In brief, in the third embodiment, because the epilepsy seizure state reflected by each EEG signal segment on the first epilepsy patient is known, the establishing module 144_7 may use the epilepsy seizure state corresponding to each EEG signal segment of the first epilepsy patient and the specific statistical feature values corresponding thereto as training data for the SVM (e.g., v-SVM). Subsequently, the establishing module 114_7 may locate a hyperplane for discriminating the epilepsy seizure state based on the EEG signal segment of the first epilepsy patient.

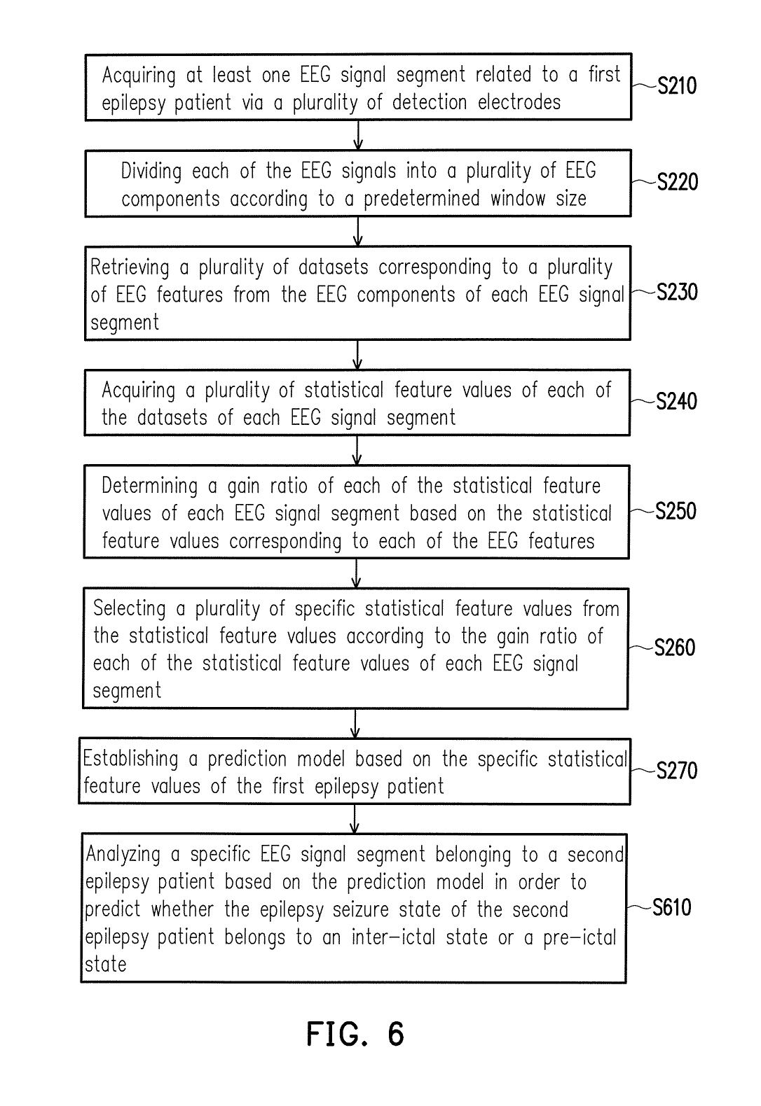

Referring to FIG. 6, which is a flowchart illustrating a method for predicting the epilepsy seizure state based on the prediction model according to an embodiment of the invention. In the present embodiment, after steps S210 to S270 are executed, in step S610, the prediction module 114_8 may analyze a specific EEG signal segment belonging to a second epilepsy patient based on the prediction model in order to predict whether the epilepsy seizure state of the second epilepsy patient belongs to an inter-ictal state or a pre-ictal state.

Specifically, the prediction module 114_8 may locate the specific statistical feature values of the second epilepsy patient based on the above teachings Subsequently, the prediction module 114_8 may input the specific statistical feature values to the prediction model in order to classify specific statistical feature values through the hyperplane in the prediction model. Thereafter, the prediction module 114_8 may predict whether the second epilepsy patient belongs to the inter-ictal state or the pre-ictal state based on a classified result.

In brief, the electronic apparatus proposed in the embodiments of the invention is capable of locating the specific statistical feature values contributing in determining the epilepsy seizure state from the EEG of the first epilepsy patient whose epilepsy seizure state is known, and establishing the prediction model for predicting the epilepsy seizure state based on the specific statistical feature values. In other words, the electronic apparatus proposed according to the embodiments of the invention provides an effective and quantized method for predicting the epilepsy seizure state.

In summary, the electronic apparatus proposed according to the embodiments of the invention is capable of establishing the prediction model for predicting the condition feature of the patient based on the EEG of the epilepsy patient. In brief, the electronic apparatus proposed in the embodiments of the invention is capable of locating the specific statistical feature values contributing in determining the condition feature from the EEG of the first epilepsy patient whose condition feature is known, and establishing the prediction model for predicting the condition feature based on the specific statistical feature values. In other words, the electronic apparatus proposed according to the embodiments of the invention provide an effective and quantized method for predicting the condition feature.

Although the present disclosure has been described with reference to the above embodiments, it will be apparent to one of ordinary skill in the art that modifications to the described embodiments may be made without departing from the spirit of the disclosure. Accordingly, the scope of the disclosure will be defined by the attached claims and not by the above detailed descriptions.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An electronic apparatus for establishing prediction model based on electroencephalogram (EEG), comprising:
a storage unit, recording a plurality of modules; and
a processing unit, coupled to the modules and configured to access and execute the modules, and the modules comprising:
a first acquiring module, acquiring at least one EEG signal segment related to a first epilepsy patient via a plurality of detection electrodes, wherein each of the at least one EEG signal segment comprises a plurality of EEG signals corresponding to a plurality of channels, and each of the channels is corresponding to one of a plurality of bipolar montages;
a dividing module, dividing each of the EEG signals into a plurality of EEG components according to a predetermined window size;
a retrieving module, retrieving a plurality of datasets corresponding to a plurality of EEG features from the EEG components of each of the at least one EEG signal segment;
a second acquiring module, acquiring a plurality of statistical feature values of each of the datasets of each of the at least one EEG signal segment;
a determining module, determining a gain ratio of each of the statistical feature values of each of the at least one EEG signal segment based on the statistical feature values corresponding to each of the EEG features;
a selecting module, selecting a plurality of specific statistical feature values from the statistical feature values according to the gain ratio of each of the statistical feature values of each of the at least one EEG signal segment; and
an establishing module, establishing a prediction model based on the specific statistical feature values of the first epilepsy patient.

2. The electronic apparatus according to claim 1, wherein the EEG features comprise an auto regressive modeling error, a decorrelation time, an EEG energy, an approximate entropy, a sample entropy, a mobility, a relative power of a plurality of frequency bands, a spectral edge frequency, a spectral edge power, a plurality of moments and a plurality of energy of wavelet coefficients.

3. The electronic apparatus according to claim 1, wherein each of the EEG signals comprises a plurality of sampling values acquired by the first acquiring module according to a sampling frequency, and an i^{th} dataset corresponding to a j^{th} EEG feature is characterized by:
$${F}_{ij} = [ \begin{matrix}{f}_{ij} ( 1 , 1 ) & {f}_{ij} ( 1 , 2 ) & … & {f}_{ij} ( 1 , {n}_{i}^{′} ) \\ {f}_{ij} ( 2 , 1 ) & {f}_{ij} ( 2 , 2 ) & … & {f}_{ij} ( 2 , {n}_{i}^{′} ) \\ ⋮ & ⋮ & ⋱ & ⋮ \\ {f}_{ij} ( C , 1 ) & {f}_{ij} ( C , 2 ) & … & {f}_{ij} ( C , {n}_{i}^{′} )\end{matrix} ] , j = 1 , … , {E}_{f}$$
wherein C is an amount of the channels, E_{f} s an amount of the EEG features, and fᵢⱼ(l,k) is a feature value of a k^{th} EEG component of a l^{th} channel,
wherein n′ᵢ=└nᵢ/(fₛ·W)┘, nᵢ is an amount of the sampling values, fₛ is the sampling frequency, W is the predetermined window size, and └•┘ is a floor function.

4. The electronic apparatus according to claim 3, wherein the statistical feature values of the i^{th} dataset corresponding to the j^{th} EEG feature comprise a plurality of average values, a plurality of standard deviations and a plurality of signal-to-noise ratios, and the second acquiring module is configured for:
calculating a plurality of inter-channel average values, a plurality of inter-channel standard deviations and a plurality of inter-channel signal-to-noise ratios of the i^{th} dataset corresponding to the j^{th} EEG feature; and
calculating the average values, the standard deviations and the signal-to-noise ratios according to the inter-channel average values, the inter-channel standard deviations and the inter-channel signal-to-noise ratios,
wherein a k^{th} inter-channel average value among the inter-channel average values is characterized by:
$${AVG}_{k} ( {F}_{ij} ) = \frac{1}{C} {\sum }_{l = 1}^{C} {f}_{ij} ( l , k ) ,$$
wherein a k^{th} inter-channel standard deviation among the inter-channel standard deviations is characterized by:
$${STD}_{k} ( {F}_{ij} ) = \sqrt{\frac{1}{C} {\sum }_{l = 1}^{C} {( {f}_{ij} ( l , k ) - {AVG}_{k} ( {F}_{ij} ) )}^{2}} ,$$
wherein a k^{th} inter-channel signal-to-noise ratio among the inter-channel signal-to-noise ratios is characterized by:
$${SNR}_{k} ( {F}_{ij} ) = \frac{{AVG}_{k} ( {F}_{ij} )}{{STD}_{k} ( {F}_{ij} )} ,$$
wherein a first average value, a second average value and a third average value among the average values are respectively characterized by:
$$avg\_AVG ( {F}_{ij} ) = \frac{1}{{n}_{i}^{′}} {\sum }_{k = 1}^{{n}_{i}^{′}} {AVG}_{k} ( {F}_{ij} ) , \\ avg\_STD ( {F}_{ij} ) = \frac{1}{{n}_{i}^{′}} {\sum }_{k = 1}^{{n}_{i}^{′}} {STD}_{k} ( {F}_{ij} ) , and$$
$$avg\_SNR ( {F}_{ij} ) = \frac{1}{{n}_{i}^{′}} {\sum }_{k = 1}^{{n}_{i}^{′}} {SNR}_{k} ( {F}_{ij} ) ,$$
wherein a first standard deviation, a second standard deviation and a third standard deviation among the standard deviations are respectively characterized by:
$${std\_AVG}_{k} ( {F}_{ij} ) = \sqrt{\frac{1}{{n}_{i}^{′}} {\sum }_{k = 1}^{{n}_{i}^{′}} {( {AVG}_{k} ( {F}_{ij} ) - avg\_AVG ( {F}_{ij} ) )}^{2}} , \\ {std\_STD}_{k} ( {F}_{ij} ) = \sqrt{\frac{1}{{n}_{i}^{′}} {\sum }_{k = 1}^{{n}_{i}^{′}} {( {STD}_{k} ( {F}_{ij} ) - avg\_STD ( {F}_{ij} ) )}^{2}} , and$$
$${std\_SNR}_{k} ( {F}_{ij} ) = \sqrt{\frac{1}{{n}_{i}^{′}} {\sum }_{k = 1}^{{n}_{i}^{′}} {( {SNR}_{k} ( {F}_{ij} ) - avg\_SNR ( {F}_{ij} ) )}^{2}} ,$$
wherein a first signal-to-noise ratio, a second signal-to-noise ratio and a third signal-to-noise ratio among the signal-to-noise ratios are respectively characterized by:
$$snr\_AVG ( {F}_{ij} ) = \frac{avg\_AVG ( {F}_{ij} )}{std\_AVG ( {F}_{ij} )} , \\ snr\_STD ( {F}_{ij} ) = \frac{avg\_STD ( {F}_{ij} )}{std\_STD ( {F}_{ij} )} , and$$
$$snr\_SNR ( {F}_{ij} ) = \frac{avg\_SNR ( {F}_{ij} )}{std\_SNR ( {F}_{ij} )} .$$

5. The electronic apparatus according to claim 4, wherein the selecting module is configured for:
ranking the gain ratio of each of the statistical feature values in a descending order according to the gain ratio of each of the statistical feature values; and
selecting a predetermined number of the top-ranked statistical feature values from the statistical feature values of each of the at least one EEG signal segment to serve as the specific statistical feature values.

6. The electronic apparatus according to claim 1, wherein the first epilepsy patient is not yet received an antiepileptic drug treatment, and an epilepsy type of the first epilepsy patient belongs to a well-controlled epilepsy or a refractory epilepsy,
wherein the modules further comprise a prediction module, configured for analyzing a specific EEG signal segment belonging to a second epilepsy patient based on the prediction model in order to predict whether the epilepsy type of the second epilepsy patient belongs to the well-controlled epilepsy or the refractory epilepsy.

7. The electronic apparatus according to claim 1, wherein a first EEG signal segment in the at least one EEG signal segment is corresponding to an EEG state of the first epilepsy patient before receiving a music therapy, and a second EEG signal segment in the at least one EEG signal segment is corresponding to the EEG state of the first epilepsy patient receiving the music therapy.

8. The electronic apparatus according to claim 7, wherein each of the EEG signals comprises a plurality of sampling values acquired by the first acquiring module according to a sampling frequency, and an i^{th} dataset corresponding to a k^{th} EEG signal segment and a j^{th} EEG feature is characterized by:
$${F}_{ij}^{( k )} = [ \begin{matrix}{f}_{ij}^{( k )} ( 1 , 1 ) & {f}_{ij}^{( k )} ( 1 , 2 ) & … & {f}_{ij}^{( k )} ( 1 , {n}_{i}^{( k )} ) \\ {f}_{ij}^{( k )} ( 2 , 1 ) & {f}_{ij}^{( k )} ( 2 , 2 ) & … & {f}_{ij} ( 2 , {n}_{i}^{( k )} ) \\ ⋮ & ⋮ & ⋱ & ⋮ \\ {f}_{ij}^{( k )} ( C , 1 ) & {f}_{ij}^{( k )} ( C , 2 ) & … & {f}_{ij}^{( k )} ( C , {n}_{i}^{( k )} )\end{matrix} ] , j = 1 , … , {E}_{f} , k = 1 , 2$$
wherein C is an amount of the channels, E_{f} is an amount of the EEG features, and fᵢⱼ^{(k)}(l,m) is a feature value of a m^{th} EEG component of a l^{th} channel,
wherein nᵢ^{(k)}=└nᵢ/(fₛ·W)┘, nᵢ is an amount of the sampling values, fₛ is the sampling frequency, W is the predetermined window size, and └•┘ is a floor function.

9. The electronic apparatus according to claim 8, wherein the statistical feature values of the i^{th} dataset corresponding to the j^{th} EEG feature comprise a plurality of average values, a plurality of standard deviations and a plurality of signal-to-noise ratios, and the second acquiring module is configured for:
calculating a plurality of first inter-channel average values, a plurality of first inter-channel standard deviations and a plurality of first inter-channel signal-to-noise ratios of the i^{th} dataset corresponding to the first EEG signal segment and the j^{th} EEG feature, and calculating a plurality of first average values, a plurality of first standard deviations and a plurality of first signal-to-noise ratios according to the first inter-channel average values, the first inter-channel standard deviations and the first inter-channel signal-to-noise ratios;
calculating a plurality of second inter-channel average values, a plurality of second inter-channel standard deviations and a plurality of second inter-channel signal-to-noise ratios of the i^{th} dataset corresponding to the second EEG signal segment and the j^{th} EEG feature, and calculating a plurality of second average values, a plurality of second standard deviations and a plurality of second signal-to-noise ratios according to the second inter-channel average values, the second inter-channel standard deviations and the second inter-channel signal-to-noise ratios;
characterizing the first average values, the first standard deviations and the first signal-to-noise ratios by a first matrix;
characterizing the second average values, the second standard deviations and the second signal-to-noise ratios by a second matrix; and
subtracting the first matrix from the second matrix in order to acquire a third matrix comprising the average values, the standard deviations and the signal-to-noise ratios.

10. The electronic apparatus according to claim 9, wherein the first epilepsy patient is a first-type patient or a second-type patient, wherein the first-type patient represents patients whose epilepsy condition is improvable by the music therapy, and the second-type patient represents patients whose epilepsy condition is not improvable by the music therapy,
wherein the modules further comprise a prediction module, configured for analyzing a specific EEG signal segment belonging to a second epilepsy patient based on the prediction model in order to predict whether the second epilepsy patient belongs to the first-type patient or the second-type patient.

11. The electronic apparatus according to claim 1, wherein the first acquiring module acquires the at least one EEG signal segment from an artifact-free signal based on a sliding window mechanism, adjacent two EEG signal segments in the at least one EEG signal segment overlap with each other for a predetermined time interval, and the sliding window mechanism is corresponding to a sliding window size.

12. The electronic apparatus according to claim 11, wherein each of the EEG signals comprises a plurality of sampling values acquired by the first acquiring module according to a sampling frequency, and an i^{th} dataset corresponding to a j^{th} EEG signal segment and a k^{th} EEG feature is characterized by:
$${F}_{ij}^{( k )} = [ \begin{matrix}{f}_{ij}^{( k )} ( 1 , 1 ) & {f}_{ij}^{( k )} ( 1 , 2 ) & … & {f}_{ij}^{( k )} ( 1 , \frac{SW}{W} ) \\ {f}_{ij}^{( k )} ( 2 , 1 ) & {f}_{ij}^{( k )} ( 2 , 2 ) & … & {f}_{ij} ( 2 , \frac{SW}{W} ) \\ ⋮ & ⋮ & ⋱ & ⋮ \\ {f}_{ij}^{( k )} ( C , 1 ) & {f}_{ij}^{( k )} ( C , 2 ) & … & {f}_{ij}^{( k )} ( C , \frac{SW}{W} )\end{matrix} ] , \\ k = 1 , … , {E}_{f} , j = 1 , … , {M}_{i}$$
wherein C is an amount of the channels, E_{f} is an amount of the EEG features, Mᵢ is an amount of the at least one EEG signal segment, fᵢⱼ^{(k)}(l,m) is a feature value of a m^{th} EEG component of a l^{th} channel, SW is the sliding window size, and W is the predetermined window size.

13. The electronic apparatus according to claim 12, wherein an epilepsy seizure state of the first epilepsy patient belongs to an inter-ictal state or a pre-ictal state,
wherein the modules further comprise a prediction module, configured for analyzing a specific EEG signal segment belonging to a second epilepsy patient based on the prediction model in order to predict whether the epilepsy seizure state of the second epilepsy patient belongs to the inter-ictal state or the pre-ictal state.
